SEQUENCE LISTING (33 sequences)

SEQ ID NO: 1 - DNA, 32 bp, synthetic construct
  source 1..32: mol_type = other DNA
gggaattccatatgctaacaccgatatccatc

SEQ ID NO: 2 - DNA, 34 bp, synthetic construct
  source 1..34: mol_type = other DNA
gccgctcgagttatattatctttttgatttcatg

SEQ ID NO: 3 - DNA, 38 bp, synthetic construct
  source 1..38: mol_type = other DNA
gggaattccatatgtaacaaattaaattgtaaaacccc

SEQ ID NO: 4 - DNA, 34 bp, synthetic construct
  source 1..34: mol_type = other DNA
gccgctcgagatacgttctcctttttgtataaaa

SEQ ID NO: 5 - DNA, 30 bp, synthetic construct
  source 1..30: mol_type = other DNA
caaaaggagatatacatatgaaagaaaccg

SEQ ID NO: 6 - DNA, 30 bp, synthetic construct
  source 1..30: mol_type = other DNA
aatttgttaagatcgatctcgatcctctac

SEQ ID NO: 7 - DNA, 37 bp, synthetic construct
  source 1..37: mol_type = other DNA
gtagaggatcgagatcgatcttaacaaattaaattgt

SEQ ID NO: 8 - DNA, 38 bp, synthetic construct
  source 1..38: mol_type = other DNA
cggtttctttcatatgtatatctccttttgtataaaat

SEQ ID NO: 9 - DNA, 23 bp, synthetic construct
  source 1..23: mol_type = other DNA
agtaaaggagaagaacttttcac

SEQ ID NO: 10 - DNA, 27 bp, synthetic construct
  source 1..27: mol_type = other DNA
ggatcttatttgtagagctcatccatg

SEQ ID NO: 11 - DNA, 35 bp, synthetic construct
  source 1..35: mol_type = other DNA
gagctctacaaataagatccggctgctaacaaagc

SEQ ID NO: 12 - DNA, 43 bp, synthetic construct
  source 1..43: mol_type = other DNA
gtgaaaagttcttctcctttactcatatgtatatctccttttg

SEQ ID NO: 13 - DNA, 32 bp, synthetic construct
  source 1..32: mol_type = other DNA
attatctctttttatttatattatattgaaac

SEQ ID NO: 14 - DNA, 21 bp, synthetic construct
  source 1..21: mol_type = other DNA
taatagaaaaggacacagaag

SEQ ID NO: 15 - DNA, 45 bp, synthetic construct
  source 1..45: mol_type = other DNA
cgagatcgatctttgaaactaaagtttattaatttcaatataata

SEQ ID NO: 16 - DNA, 41 bp, synthetic construct
  source 1..41: mol_type = other DNA
atcctctaagctttatttatttttggctttttaaacagaac

SEQ ID NO: 17 - DNA, 34 bp, synthetic construct
  source 1..34: mol_type = other DNA
atcctctaagctttaatagaaaaggacacagaag

SEQ ID NO: 18 - DNA, 45 bp, synthetic construct
  source 1..45: mol_type = other DNA
ataaatttaattttatacaaaaggagatatacatatgagtaaagg

SEQ ID NO: 19 - DNA, 28 bp, synthetic construct
  source 1..28: mol_type = other DNA
attatattgacgccggacgcatcgtggc

SEQ ID NO: 20 - DNA, 30 bp, synthetic construct
  source 1..30: mol_type = other DNA
ccggattggctcaccacagccaggatccga

SEQ ID NO: 21 - DNA, 30 bp, synthetic construct
  source 1..30: mol_type = other DNA
cgtcccattccaattcgttcaagccgaggg

SEQ ID NO: 22 - DNA, 30 bp, synthetic construct
  source 1..30: mol_type = other DNA
gaacgaattggaatgggacgcgccctgtag

SEQ ID NO: 23 - DNA, 33 bp, synthetic construct
  source 1..33: mol_type = other DNA
gctgtggtgagccaatccggatatagttcctcc

SEQ ID NO: 24 - DNA, 20 bp, synthetic construct
  source 1..20: mol_type = other DNA
gatccggctgctaacaaagc

SEQ ID NO: 25 - DNA, 38 bp, synthetic construct
  source 1..38: mol_type = other DNA
ttgtataaaattaaatttatattatattgacgccggac

SEQ ID NO: 26 - DNA, 44 bp, synthetic construct
  source 1..44: mol_type = other DNA
aatataaatttaattttatacaagaggaacagatgaagcgaacc

SEQ ID NO: 27 - DNA, 40 bp, synthetic construct
  source 1..40: mol_type = other DNA
gctttgttagcagccggatcttaccccatgtgcaagccgc

SEQ ID NO: 28 - DNA, 533 bp, synthetic construct
  source 1..533: mol_type = other DNA
taacaaattaaattgtaaaacccctcttaagcatagttaagaggggtaggttttaaattttttgttgaaattagaaaaaataataaaaaaacaaacctattttctttcaggttgtttttgggttacaaaacaaaaagaaaacatgtttcaaggtacaataattatggttctttagctttctgtaaaacagccttaatagttggatttatgactattaaagttagtatacagcatacacaatctattgaaggatatttataatgcaattccctaaaaatagttttgtataaccagttcttttatccgaactgatacacgtattttagcataatttttaatgtatcttcaaaaacagcttctgtgtccttttctattaaacatataaattcttttttatgttatatatttataaaagttctgtttaaaaagccaaaaataaataattatctctttttatttatattatattgaaactaaagtttattaatttcaatataatataaatttaattttatacaaaaaggagaacgtat

SEQ ID NO: 29 - DNA, 509 bp, synthetic construct
  source 1..509: mol_type = other DNA
taacaaattaaattgtaaaacccctcttaagcatagttaagaggggtaggttttaaattttttgttgaaattagaaaaaataataaaaaaacaaacctattttctttcaggttgtttttgggttacaaaacaaaaagaaaacatgtttcaaggtacaataattatggttctttagctttctgtaaaacagccttaatagttggatttatgactattaaagttagtatacagcatacacaatctattgaaggatatttataatgcaattccctaaaaatagttttgtataaccagttcttttatccgaactgatacacgtattttagcataatttttaatgtatcttcaaaaacagcttctgtgtccttttctattaaacatataaattcttttttatgttatatatttataaaagttctgtttaaaaagccaaaaataaataattatctctttttatttatattatattgaaactaaagtttattaatttcaatataatataaatttaa

SEQ ID NO: 30 - DNA, 600 bp, synthetic construct
  source 1..600: mol_type = other DNA
taacaaattaaattgtaaaacccctcttaagcatagttaagaggggtaggttttaaattttttgttgaaattagaaaaaataataaaaaaacaaacctattttctttcaggttgtttttgggttacaaaacaaaaagaaaacatgtttcaaggtacaataattatggttctttagctttctgtaaaacagccttaatagttggatttatgactattaaagttagtatacagcatacacaatctattgaaggatatttataatgcaattccctaaaaatagttttgtataaccagttcttttatccgaactgatacacgtattttagcataatttttaatgtatcttcaaaaacagcttctgtgtccttttctattaaacatataaattcttttttatgttatatatttataaaagttctgtttaaaaagccaaaaataaataattatctctttttatttatattatattgaaactaaagtttattaatttcaatataatataaatttaattttatacaaaaggagatatacatatgagtaaaggagaagaacttttcactggagttgtcccaattcttgttgaattagatggtgatgtta

SEQ ID NO: 31 - protein, 22 aa, synthetic construct
MSKGEELFTGVVPILVELDGDV

SEQ ID NO: 32 - DNA, 25 bp, synthetic construct
  source 1..25: mol_type = other DNA
attatctctttttatttatattata

SEQ ID NO: 33 - DNA, 25 bp, synthetic construct
  source 1..25: mol_type = other DNA
aagcttagaggatcgagatcgatct

(19) United States
(12) Patent Application Publication
(10) Pub. No.: US 2023/0416762 A1
(43) Pub. Date: Dec. 28, 2023

(54) RECOMBINANT PLASMID FOR MANUFACTURING BIO-BASED MATERIALS

(71) Applicant: NATIONAL CHUNG HSING UNIVERSITY, Taichung City (TW)

(72) Inventors: Si-Yu Li, Taichung City (TW); Ying-Xing Liu, Taichung City (TW); Xiao-Zhen Zhuo, Taichung City (TW); Yu-Ning Wang, Taichung City (TW)

(21) Appl. No.: 18/209,726

(22) Filed: Jun. 14, 2023

Related U.S. Application Data
(60) Provisional application No. 63/351,824, filed on Jun. 14, 2022.

(51) Int. Cl. C12N 15/64

(52) U.S. Cl. CPC C12N 15/64

(57) ABSTRACT

The present invention provides a recombinant plasmid for manufacturing bio-based materials, comprising: a promoter system; and a rbs′-synthetic gene cluster, wherein the promoter system comprises a Pribnow box and a transcriptional activator.

### CROSS-REFERENCE TO RELATED APPLICATION

This application claims the benefit under 35 U.S.C. § 119(e) to the U.S. provisional patent application having the Ser. No. 63/351,824 filed on Jun. 14, 2022, the entirety of which is incorporated herein by reference.

### STATEMENT REGARDING SEQUENCE LISTING

The sequence listing associated with this application is provided in xml format is hereby incorporated by reference into the specification. The name of the XML file containing the sequence listing is 69720-012.xml. The XML file is 27,981 bytes; was created on Sep. 13 2023; and is being submitted electronically via Patent center.

### FIELD OF INVENTION

The present invention relates to a recombinant plasmid for manufacturing bio-based materials. In particular, it relates to recombinant plasmid including a newly developed promoter system for manufacturing bio-based materials.

### BACKGROUND OF THE INVENTION

A promoter is a cis-acting DNA sequence recognized by RNA polymerase (RNAP) for subsequent transcription. Promoter activity often involves the interaction of the promoter with RNAP and regulatory factors, including regulatory proteins (activator and repressor) and effector molecules (inducer and corepressor). Promoter activity can be quantified by the probability of promoter occupancy by RNA polymerase based on thermodynamic models, and the probability of promoter occupancy by RNAP being proportional to the gene expression is one of the most important assumptions in these models. Transcription regulation can be negative inducible or negative repressible when the repressor protein cooperates with inducers or corepressor, respectively. Additionally, the transcription regulation can be positive inducible or positive repressible when the activator protein cooperates with inducers or corepressors, respectively. Transcriptional regulation usually occurs at the transcription binding and initiation stages.

Bacillus anthracis is a gram-positive, aerobic, and spore-forming bacillus. B. anthracis, which carries two virulence plasmids, pXO1 (182 kb) and pXO2 (95 kb), is a virulent strain. The first mega plasmid pXO1 carries three virulent genes pagA, lef, and cya [encoding cell-binding protective antigen (PA, 85 kDa), lethal factor (LF, 83 kDa), and edema factor (EF, 89 kDa)]. A pair of PA and EF proteins forms an edema toxin and that of PA and LF proteins forms a lethal toxin. The mega plasmid pXO2 carries the capBCADE operon for the production of the antiphagocytic poly-D-glutamic acid capsule, protecting the pathogen from phagocytosis. The secretion of toxins and capsules from B. anthracis is considered critical to protect it against the host immune system and to cause infection. The expression of these virulence genes is highly regulated at the transcriptional initiation level by a class of regulators called phosphoenolpyruvate-dependent phosphotransferase regulation domain containing virulence regulators (PCVRs).

Further, anthrax toxin activator (AtxA, encoded on pXO1) is a major positive regulator of anthrax toxin and capsule expression. Thus, it is a potential target for developing therapeutics for anthrax infection. AtxA is a PCVR that contains two helix-turn-helix (HTH) domains at the N-terminus, two phosphotransferase system regulation domains (PRDs), and one EIIB-like domain at the C-terminus. It has been suggested that the homodimeric state of AtxA is the active structure for regulation, and the dimerization of AtxA is positively promoted by a high CO₂/bicarbonate level, dephosphorylation of the EIIB domain and H379 in PRD2. Furthermore, the phosphorylation of H199 in PRD1 is essential for DNA binding. All three functional domains of AtxA are commonly found in PCVR; nevertheless, the molecular mechanisms of AtxA in regulating toxin expression are not fully clear.

The pagA gene has two promoters, P1 and P2. The transcription start sites (TSSs) of P1 and P2 transcripts are −58 and −26 relative to the translation initiation codon site of PA. P1 is regulated by AtxA and the 90 base pair (bp) DNA region upstream of the P1 TSS is sufficient for AtxA-dependent transcription regulation. The sequence of the 90-bp DNA region upstream of the P1 TSS is high in AT % and is predicted to form an intrinsic AT_{rich} curvature structure. The AtxA specific regulation is DNA-structure specific rather than sequence-specific. In some researches, it was further demonstrated that (−105)-(−67) upstream of the P1 TSS is an essential cis-acting site where the region forms a stem loop structure (and thus named as SLII). The specific interaction between AtxA and SLII is thought to be structure-specific rather than sequence-specific. A second curvature is predicted 30 bp downstream of the P1 TSS, and it has been shown that the second curvature is not necessary for AtxA-dependent transcription regulation. Some research have been reported for AtxA-dependent transcription; however, the exact sequence of the P1 promoter has not been fully elucidated. In contrast, P2 is thought to be a constitutive and relatively weak promoter. The −10 and −35 sequences have been suggested, despite the fact that the predicted −35 sequence, TTCCCA, of P2 differs from the consensus sequence, TTGACA and the space of 20 bp between the predicted −10 and −35 sequence is not optimal. A recent RNA-seq analysis disclosed that transcripts starting from P2 are abundant.

The above information disclosed in this section is only for enhancement of understanding of the background of the described technology and therefore it may contain information that does not form the prior art that is already known to a person of ordinary skill in the art.

### SUMMARY OF THE INVENTION

In the present invention, a 509-bp DNA fragment [(−508)-(+1) relative to the P2 TSS), and termed 509 sequence in the present invention], containing P1 and P2, was cloned upstream of rbs-GFPuv to elucidate the AtxA-based transcription regulation. First, the 509 sequence was dissected into five genetic elements: −10 sequence, −35 sequence, Airier, tract, SLI/SLII, and the upstream site (FIG. 1). The regulatory role of each cis-acting element in promoter activity was systematically investigated in conjunction with the heterologous co-expression of AtxA in E. coli. While P2 is thought to be a weak and constitutive promoter in B. anthracis, the present invention is the first to prove that AtxA-regulated transcription can be independent from P1 and P2. By understanding the interaction among the cis-acting site, cognate RNAP, and AtxA, a novel promoter three-hybrid, P3H_{-10,AtxA}, has been first demonstrated, where the hybrid talks of −10 sequence (TATACT or Pribnow box), AtxA, and host RNAP provides a tight-control, high-level, modulable, and stationary-phase specific expression promoter. Furthermore, the phaCAB genes of C. manganoxidans were cloned downstream of P3H-10, AtxA and the stationary-phase PHB production in E. coli was examined and reported.

To achieve aforementioned effect, the present invention provides a recombinant plasmid for manufacturing bio-based materials, comprising: a promoter system; and a rbs′-synthetic gene cluster, wherein the promoter system comprises a Pribnow box and a transcriptional activator.

In one embodiment of the present invention, the method of producing the promoter system comprises: (a) inserting a 509seq (SEQ ID NO: 29) into pET29a-EGFP to form a plasmid pTOL02A; (b) replacing EGFP gene of pTOL02A with the GFPuv gene to form the pTOL02B; (c) deleting a SLI/SLII from the plasmid pTOL02B to form a plasmid pTOL02C; (d) subjecting the plasmid pTOL02C to site-directed mutagenesis deletion to form a plasmid pTOL02F; and (e) inserting the transcriptional activator into the plasmid pTOL02F to form a plasmid pTOL03F.

In one embodiment of the present invention, comprising replacing an rbs-GFPᵤᵥ of the plasmid pTOL03F with the rbs′-synthetic gene cluster, and the rbs′-synthetic gene cluster comprises a rbs′-phaCAB gene cluster.

In one embodiment of the present invention, the site-directed mutagenesis deletion of the step (d) deletes −35, AT_{rich} tract, and an upstream site of the plasmid pTOL02C.

In one embodiment of the present invention, the transcriptional activator is an AtxA operon.

In one embodiment of the present invention, the AtxA operon is a P_{T7}-rbs-atxA-T7 terminator.

In one embodiment of the present invention, the bio-based materials comprises polyhydroxybutyrate.

In one embodiment of the present invention, an activity of the promoter system is regulated by the transcriptional activator, a Pribnow box, and a cognate RNAP.

Many of the attendant features and advantages of the present invention will become better understood with reference to the following detailed description considered in connection with the accompanying drawings.

### BRIEF DESCRIPTION OF THE DRAWINGS

The detailed structure, operating principle and effects of the present invention will now be described in more details hereinafter with reference to the accompanying drawings that show various embodiments of the present invention as follows.

FIG. 1 (A) Schematic of pTOL02 series harboring genetic elements in 509seq. (B) Nucleotide sequences of the 509seq with labeled genetic elements. (C) Constructions of pTOL03 series by inserting the AtxA operon in pTOLAtxA into pTOL02 series. The plasmid sizes of pTOL02D, pTOL02E, and pTOL02F are 6,359, 6,293, and 5,850 bp, respectively. The plasmid sizes of pTOL03D, pTOL03E, and pTOL03F are 8,312, 8,246, and 7,803 bp, respectively.

FIG. 2 illustrates the specific fluorescence intensity of E. coli BL21(DE3) strains harboring pTOL02B, pTOLAtxA, or pTOL02B/pTOLAtxA combination. The control represents E. coli BL21(DE3) and (−) symbol represents no IPTG induction while the (+) symbol represents the IPTG supplementation to the final conc. of 0.03 mM at the cultivation time of 2 h. The bacterial samples were collected at cultivation time of 12 h for fluorescence intensity measurements, where OD₆₀₀ values were in the range between 3.5 and 4.5. Errors represent standard deviation with n=3.

FIG. 3 illustrates the specific fluorescence intensity of E. coli BL21(DE3) strains harboring pTOL02C, pTOL02C/pTOLAtxA, pTOL02D, pTOL02D/pTOLAtxA, pTOL02E, or pTOL02E/pTOLAtxA. All strains were subjected to the IPTG supplementation to the final conc. of 0.03 mM at the cultivation time of 2h. The bacterial samples were collected at cultivation time of 12 h for fluorescence intensity measurements, where OD₆₀₀ values were in the range between 3.5 and 4.5. Errors represent standard deviation with n=3 for pTOL02C, pTOL02C+pTOLAtxA; and n=15 for pTOL02D, pTOL02D/pTOLAtxA, pTOL02E, and pTOL02E/pTOLAtxA.

FIG. 4 illustrates the specific fluorescence intensity of E. coli BL21(DE3) strains harboring pTOL02F and pTOL02F/pTOLAtxA. All strains were subjected to the IPTG supplementation to the final conc. of 0.03 mM at the cultivation time of 2 h. The bacterial samples were collected at cultivation time of 12 h for fluorescence intensity measurements. Errors represent standard deviation with n=6 for pTOL02F, n=15 for pTOL02F+AtxA, and n=3 for pTOL03F.

FIG. 5 illustrates the effect of NaCl concentration on the (A) bacterial growth and (B) specific fluorescence intensity of E. coli BL21(DE3)/pTOL03D. The induction of AtxA expression was achieved with the 30 μM IPTG when OD₆₀₀ of bacterial cultures reached (−0.9 and −0.5 on the log scale). Errors represent standard deviation with n=6.

FIG. 6 illustrates the effect of induction time on (A) the growth and (B) the specific fluorescence intensity of E. coli BL21(DE3)/pTOL02D+pTOLAtxA. Final IPTG concentration of 30 nM was used for the induction of AtxA expression. Errors represent standard deviation with n=6.

FIG. 7 illustrates the dependence of specific fluorescence intensity of (A) pTOL03E and (B) pTOL03F on the AtxA induction, which was driven by IPTG. The supplementation of IPTG was achieved when OD₆₀₀ of bacterial cultures reached 0.4-0.6. Errors represent standard deviation with n=3.

FIG. 8 illustrates PHB production in E. coli BL21P/pTOL03FCAB. E. coli BL21P/pTOL03FCAB was first aerobically cultivated in LB for 6 h, followed by the supplementation of glucose and IPTG to reach the final concentrations of 10 g/L and 15 nM, respectively. Errors represent standard deviation with n=3.

### DETAILED DESCRIPTION OF THE PREFERRED EMBODIMENTS

Reference will now be made in detail to the exemplary embodiments of the present invention, examples of which are illustrated in the accompanying drawings. Therefore, it is to be understood that the foregoing is illustrative of exemplary embodiments and is not to be construed as limited to the specific embodiments disclosed, and that modifications to the disclosed exemplary embodiments, as well as other exemplary embodiments, are intended to be included within the scope of the appended claims. These embodiments are provided so that this invention will be thorough and complete, and will fully convey the inventive concept to those skilled in the art.

For convenience, certain terms employed in the specification, examples and appended claims are collected here. Unless defined otherwise, all technical and scientific terms used herein have the same meaning as commonly understood by one of the ordinary skill in the art to which this invention belongs.

Various embodiments will now be described more fully with reference to the accompanying drawings, in which illustrative embodiments are shown. The inventive concept, however, may be embodied in various different forms, and should not be construed as being limited only to the illustrated embodiments. Rather, these embodiments are provided as examples, to convey the inventive concept to one skilled in the art. Accordingly, known processes, components, and techniques are not described with respect to some of the embodiments.

The singular forms “a”, “and”, and “the” are used herein to include plural referents unless the context clearly dictates otherwise.

The biological materials used in the present invention need not be deposited according to 37 CFR 1.802 since those biological materials are known and available to the public, or can be made, or isolated without undue experimentation.

It should be noted that, the product in the present invention includes, but not limited to polyhydroxybutyrate. Any bio-based materials that can be theoretically produced by the process of the present invention can be the products in the present invention. Similarly, rbs′-phaCAB gene cluster used in the present invention is merely one kind of the gene cluster to replace an rbs-GFPᵤᵥ, and any other synthetic gene cluster that is suitable for the process of the present invention can replace phaCAB in the present invention.

The following descriptions are provided to elucidate a method of creating a recombinant microorganism for manufacturing a fermentation product and to aid it of skilled in the art in practicing this invention. These embodiments are merely exemplary embodiments and in no way to be considered to limit the scope of the invention in any manner.

Material and Method

Construction of pTOLAtxA, pCDFDuet-533seq, and pTOL02A

The method of constructing pTOLAtxA, pCDFDuet-533seq, and pTOL02A in the present invention is as follows. The recombinant pTOLAtxA was constructed by inserting the PCR-generated atxA fragment (NC_007322.2) into pCDFDuet-1 at Ndel and Xhol sites. The primers dlc-F-AtxA-01 (SEQ ID NO: 1) and dlc-R-AtxA-01 (SEQ ID NO: 2) were used for generating atxA fragment. The sequences of primers used in the present invention are shown in Table 2. The recombinant pCDFDuet-533seq was constructed by inserting the PCR-generated 533-bp DNA fragment [(−508)-(+25) relative to the P2 TSS)] (SEQ ID NO: 28) to pCDFDuet-1 at Ndel and Xhol sites. The primers dlc-F-cisacting-01 (SEQ ID NO: 3) and dlc-R-cisacting-01 (SEQ ID NO: 4) were used for generating the 533-bp DNA fragment. The recombinant pTOL02A was constructed by inserting the PCR-generated 509-bp DNA fragment [(−508)-(+1) relative to the P2 TSS)] (SEQ ID NO: 29) into pET29a-EGFP backbone. The vector backbone was generated by PCR using pET29a-EGFP as the template and primers of SLIC-F-pET29a-GFP-01 (SEQ ID NO: 5) and SLIC-R-pET29a-GFP-01 (SEQ ID NO: 6). The insert 509-bp DNA fragment was generated by PCR using pCDFDuet-533seq as the template and primers of SLIC-F-cisacting-02 (SEQ ID NO: 7) and SLIC-R-cisacting-02 (SEQ ID NO: 8). The sequences of aforementioned primers are list in Table 2.

Construction of pTOL02 and pTOL03 Series

To construct pTOL02B, the DNA fragment encoding full-length GFPuv was amplified from pDSKGFP. with primers SLIC-F-GFP.-01 (SEQ ID NO: 9) and SLIC-R-GFP.-01 (SEQ ID NO: 10), and the vector fragment was amplified from pTOL02A, a pET29a-based backbone, with primers SLIC-F-pTOL02A-01 (SEQ ID NO: 11) and SLIC-R-pTOL02A-01 (SEQ ID NO: 12). The assembly of two fragments was achieved by one-step sequence- and ligation-independent cloning (SLIC), which is a conventional technology. In brief, the vector and insert fragments in deionized water were mixed at molar ratios of 1:1. One microliter of T4 DNA polymerase (New England BioLabs Inc., MA, USA) was added to 10 μL of the vector/insert mixture and incubated at room temperature for 2.5 min. The reaction mixture was immediately placed in an ice bath for 10 min to inhibit the nuclease reaction while facilitating annealing. This mixture was directly used for the bacterial transformation. pTOL02C was obtained by deleting SLI/SLII from pTOL02B using the Q5 ® Site-Directed Mutagenesis Kit (New England BioLabs Inc., MA, USA) with primers DL-F-SLI/II-01 (SEQ ID NO: 13) and DL-R-SLI/II-01 (SEQ ID NO: 14). The linearized pTOL02B fragment containing the SLI/SLII deletion mutation was generated by polymerase chain reaction (PCR) with pTOL02B as the template and DL-F-SLI/II-01 (SEQ ID NO: 13) and DL-R-SLI/II-01 (SEQ ID NO: 14) as the primer set. Then, 1 μL of PCR product was mixed with the kinase, ligase, and Dpnl (KLD) enzyme mixture (New England BioLabs Inc., MA, USA) to circulize the linearized DNA fragment while removing the pTOL02B template.

pTOL02D and pTOL02E were constructed by substitution mutations of pTOL02B and pTOL02C, respectively, using the Q5 ® Site-Directed Mutagenesis Kit (New England BioLabs Inc., MA, USA) as described above. The primer sets of SS-F-ATGC average-01 (SEQ ID NO: 15)/SS-R-ATGC average-01 (SEQ ID NO: 16) and SS-F-ATGC average-01 (SEQ ID NO: 15)/SS-R-ATGC average-02 (SEQ ID NO: 17) were used for pTOL02D and pTOL02E constructions, respectively. The AT_{rich} tract (ATTATCTCTTTTTATTTATATTATA, 92% AT) in pTOL02B and pTOL02C was replaced with a random-sequence DNA fragment, ATCG_{avg} (AAGCTTAGAGGATCGAGATCGATCT, 56% AT).

The recombinant plasmid pTOL02F was prepared by subjecting pTOL02E to site-directed mutagenesis deletion (Q5 ® Site-Directed Mutagenesis Kit, New England BioLabs Inc., MA, USA) with primers DL-F- −10 upstream-01 (SEQ ID NO: 18)/DL-R- −10 upstream-01 (SEQ ID NO: 19), so that −35 and the upstream site was removed. The resulting pTOL02F contained 4% of the 509 sequence and only the −10 sequence was retained.

The pTOL03 series was obtained by combining the synthetic atxA operon (P_{T7}-rbs-atxA-T7 terminator) in the pTOLAtxA with pTOL02 series. The synthetic atxA operon (1,953 bp) was obtained by PCR using primers HF-F-AtxA-01 (SEQ ID NO: 20)/HF-R-AtxA-01 (SEQ ID NO: 21), and the pTOL02 backbone was obtained by PCR with primers HF-F-pTOL02system-01 (SEQ ID NO: 22)/HF-R-pTOL02system-01 (SEQ ID NO: 23). The synthetic atxA operon and the pTOL02 backbone were assembled using the NEBuilder HiFi DNA Assembly Kit (New England BioLabs Inc., MA, USA).

pTOL03FphaCAB was obtained by replacing the rbs-GFPuv in pTOL03F with rbs′-phaCAB gene cluster of C. manganoxidans. The rbs′-phaCAB gene cluster (3,725 bp) was obtained by PCR using primers HF-F-phaCAB-01 (SEQ ID NO: 26)/HF-R-phaCAB01 (SEQ ID NO: 27), and the pTOL03F backbone was obtained by PCR with primers HF-F-pTOL03system-01 (SEQ ID NO: 24)/HF-R-pTOL03system-01 (SEQ ID NO: 25). The rbs' phaCAB gene cluster and the pTOL03F backbone were assembled using the NEBuilder HiFi DNA Assembly Kit (New England BioLabs Inc., MA, USA). The rbs' sequence is the native rbs sequence of phaCAB operon in C. manganoxidans.

Recombinant plasmids were constructed and maintained in E. coli DH5α. Plasmid DNA was isolated from E. coli was by using the Miniprep Purification Kit (QIAGEN, Hilden, Germany) Electroporation (Bio-Rad Gene Pulser, Bio-Rad, California, USA) was used for transformation.

All the plasmids used in the present invention, and the sources are listed in Table 1 as follows.

TABLE 1
List of bacterial strains and plasmids used in the present invention

| Plasmids | Descriptions | Reference |
|---|---|---|
| pTOLAtxA | Recombinant plasmid carries atxA (derived from | The present |
| | Bacillus anthracis) under the control of T7 promoter, | invention |
| | where atxA was cloned at NdeI and XhoI in | |
| | pCDFDuet-1 vector. | |
| pCDFDuet-533seq | Recombinant plasmid carries a 533-bp DNA fragment | Available |
| | [(−508)-(+25) relative to the P2 TSS)], derived from | from Dr. |
| | Bacillus anthracis], where the 533-bp DNA fragment | Jyh-hwa |
| | was cloned at NdeI and XhoI in pCDFDuet-1 vector. | Kau |
| pET29a-EGFP | Recombinant plasmid carries EGFP gene (derived | Available |
| | from Aequorea victoria) under the control of the T7 | from Dr. |
| | promoter. | Menghsiao |
| | | Meng |
| pDSK-GFPᵤᵥ | Recombinant plasmid carries the GFPuv gene under | Available |
| | the control of P_{PsbA}. pDSK519 backbone. | from Dr. |
| | | Menghsiao |
| | | Meng |
| pTOL02A | pET29a-EGFP derived recombinant plasmid where the | The present |
| | T7 promoter is replaced with the 509seq. | invention |
| pTOL02B | pTOL02A derived recombinant plasmid where the | The present |
| | EGFP gene is replaced with the GFPᵤᵥ gene. | invention |
| pTOL02C | pTOL02B derived recombinant plasmid carrying the | The present |
| | SLI/SLII deletion. | invention |
| pTOL02D | pTOL02B derived recombinant plasmid carrying with | The present |
| | the substitution mutation ΔAT_{rich}::ATCG_{average}. AT_{rich} is | invention |
| | a 25-bp sequence. | |
| pTOL02E | pTOL02B derived recombinant plasmid carrying the | The present |
| | SLI/SLII deletion and substitution mutation | invention |
| | ΔAT_{rich}::ATCG_{average}. | |
| pTOL02F | pTOL02B derived recombinant plasmid carrying the | The present |
| | gene deletion of the upstream site, SLI/SLII, AT_{rich} | invention |
| | tract, and −35 sequence. | |
| pTOL03D | pTOL02D derived, inserting the synthetic atxA operon | The present |
| | (PT7-rbs-atxA-T7 terminator) of pTOLAtxA into the | invention |
| | downstream of GFPᵤᵥ of pTOL02D. | |
| pTOL03E | pTOL02E derived, inserting the synthetic atxA operon | The present |
| | (PT7-rbs-atxA-T7 terminator) of pTOLAtxA into the | invention |
| | downstream of GFPᵤᵥ of pTOL02E. | |
| pTOL03F | pTOL02F derived, inserting the synthetic atxA operon | The present |
| | (PT7-rbs-atxA-T7 terminator) of pTOLAtxA into the | invention |
| | downstream of GFPᵤᵥ of pTOL02F. | |
| pTOL03FphaCAB | pTOL03F derived, replacing the rbs-GFPᵤᵥ with | The present |
| | rbs′-phaCAB gene cluster. | invention |

1. pCDFDuet-533seq is available from Dr. Jyh-hwa Kau, a professor of Graduate Institute of Medical Sciences at National Defense Medical Center and Institute of Preventive Medicine at National Defense Medical Center, Taiwan (ROC), for the kind gift of the 533-bap DNA fragment for constructing pCDFDuet-533seq.
2. pET29a-EGFP and pDSK-GFPuv are available from Dr. Menghsiao Meng, Professor of Graduate Institute of Biotechnology at National Chung Hsing University, Taiwan (ROC), for the kind gifts of pET29a-EGFP and pDSK-GFPuv.

TABLE 2
Primer sequence in the present invention

| | | SEQ ID |
|---|---|---|
| Primer | Sequence | NO: |
| dlc-F-AtxA-01 | GGGAATTCCATATGCTAACACCGATATCCATC | (1) |
| dlc-R-AtxA-01 | GCCGCTCGAGTTATATTATCTTTTTGATTTCATG | (2) |
| dlc-F-cisacting-01 | GGGAATTCCATATGTAACAAATTAAATTGTAAAA | (3) |
| | CCCC | |
| dlc-R-cisacting-01 | GCCGCTCGAGATACGTTCTCCTTTTTGTATAAAA | (4) |
| SLIC-F-pET29a-GFP-01 | CAAAAGGAGATATACATATGAAAGAAACCG | (5) |
| SLIC-R-pET29a-GFP-01 | AATTTGTTAAGATCGATCTCGATCCTCTAC | (6) |
| SLIC-F-cisacting-02 | GTAGAGGATCGAGATCGATCTTAACAAATTAAAT | (7) |
| | TGT | |
| SLIC-R-cisacting-02 | CGGTTTCTTTCATATGTATATCTCCTTTTGTATAA | (8) |
| | AAT | |
| SLIC-F-GFPᵤᵥ-01 | AGTAAAGGAGAAGAACTTTTCAC | (9) |
| SLIC-R-GFPᵤᵥ-01 | GGATCTTATTTGTAGAGCTCATCCATG | (10) |
| SLIC-F-pTOL02A-01 | GAGCTCTACAAATAAGATCCGGCTGCTAACAAAG | (11) |
| | C | |
| SLIC-R-pTOL02A-01 | GTGAAAAGTTCTTCTCCTTTACTCATATGTATATC | (12) |
| | TCCTTTTG | |
| DL-F-SLI/II-01 | ATTATCTCTTTTTATTTATATTATATTGAAAC | (13) |
| DL-R-SLI/II-01 | TAATAGAAAAGGACACAGAAG | (14) |
| SS-F-ATGC average-01 | CGAGATCGATCTTTGAAACTAAAGTTTATTAATTT | (15) |
| | CAATATAATA | |
| SS-R-ATGC average-01 | ATCCTCTAAGCTTTATTTATTTTTGGCTTTTTAAAC | (16) |
| | AGAAC | |
| SS-R-ATGC average-02 | ATCCTCTAAGCTTTAATAGAAAAGGACACAGAAG | (17) |
| DL-F- -10 upstream-01 | ATAAATTTAATTTTATACAAAAGGAGATATACAT | (18) |
| | ATGAGTAAAGG | |
| DL-R- -10 upstream-01 | ATTATATTGACGCCGGACGCATCGTGGC | (19) |
| HF-F-AtxA-01 | CCGGATTGGCTCACCACAGCCAGGATCCGA | (20) |
| HF-R-AtxA-01 | CGTCCCATTCCAATTCGTTCAAGCCGAGGG | (21) |
| HF-F-pTOL02system-01 | GAACGAATTGGAATGGGACGCGCCCTGTAG | (22) |
| HF-R-pTOL02system-01 | GCTGTGGTGAGCCAATCCGGATATAGTTCCTCC | (23) |
| HF-F-pTOL03system-01 | GATCCGGCTGCTAACAAAGC | (24) |
| HF-R-pTOL03system-01 | TTGTATAAAATTAAATTTATATTATATTGACGCCG | (25) |
| | GAC | |
| HF-F-phaCAB-01 | AATATAAATTTAATTTTATACAAGAGGAACAGAT | (26) |
| | GAAGCGAACC | |
| HF-R-phaCAB-01 | GCTTTGTTAGCAGCCGGATCTTACCCCATGTGCAA | (27) |
| | GCCGC | |

TABLE 3
Sequence of 533-bp DNA fragment in pCDFDuet-533seq
and 509-bp DNA fragment in pTOL02A

| | | SEQ |
|---|---|---|
| | | ID |
| Nucleotide | Sequence | NO: |
| 533-bp DNA | TAACAAATTAAATTGTAAAACCCCTCTTAAGCAT | (28) |
| fragment | AGTTAAGAGGGGTAGGTTTTAAATTTTTTGTTGAA | |
| | ATTAGAAAAAATAATAAAAAAACAAACCTATTTT | |
| | CTTTCAGGTTGTTTTTGGGTTACAAAACAAAAAG | |
| | AAAACATGTTTCAAGGTACAATAATTATGGTTCTT | |
| | TAGCTTTCTGTAAAACAGCCTTAATAGTTGGATTT | |
| | ATGACTATTAAAGTTAGTATACAGCATACACAAT | |
| | CTATTGAAGGATATTTATAATGCAATTCCCTAAA | |
| | AATAGTTTTGTATAACCAGTTCTTTTATCCGAACT | |
| | GATACACGTATTTTAGCATAATTTTTAATGTATCT | |
| | TCAAAAACAGCTTCTGTGTCCTTTTCTATTAAACA | |
| | TATAAATTCTTTTTTATGTTATATATTTATAAAAG | |
| | TTCTGTTTAAAAAGCCAAAAATAAATAATTATCT | |
| | CTTTTTATTTATATTATATTGAAACTAAAGTTTAT | |
| | TAATTTCAATATAATATAAATTTAATTTTATACAA | |
| | AAAGGAGAACGTAT | |
| 509-bp DNA | TAACAAATTAAATTGTAAAACCCCTCTTAAGCAT | (29) |
| fragment | AGTTAAGAGGGGTAGGTTTTAAATTTTTTGTTGAA | |
| | ATTAGAAAAAATAATAAAAAAACAAACCTATTTT | |
| | CTTTCAGGTTGTTTTTGGGTTACAAAACAAAAAG | |
| | AAAACATGTTTCAAGGTACAATAATTATGGTTCTT | |
| | TAGCTTTCTGTAAAACAGCCTTAATAGTTGGATTT | |
| | ATGACTATTAAAGTTAGTATACAGCATACACAAT | |
| | CTATTGAAGGATATTTATAATGCAATTCCCTAAA | |
| | AATAGTTTTGTATAACCAGTTCTTTTATCCGAACT | |
| | GATACACGTATTTTAGCATAATTTTTAATGTATCT | |
| | TCAAAAACAGCTTCTGTGTCCTTTTCTATTAAACA | |
| | TATAAATTCTTTTTTATGTTATATATTTATAAAAG | |
| | TTCTGTTTAAAAAGCCAAAAATAAATAATTATCT | |
| | CTTTTTATTTATATTATATTGAAACTAAAGTTTAT | |
| | TAATTTCAATATAATATAAATTTAA | |

### Fluorescence Assay

One milliliter of bacterial solution was collected and centrifuged at 17,000×g for 3 min at 4° C. The supernatant was decanted and the bacterial pellet was washed three times with phosphate buffered saline (137 mM NaCl, 2.7 mM KCl, 10 mM Na₂HPO₄·2H₂O, 2 mM KH₂PO₄) at 4° C. Finally, bacterial pellet was resuspended in 1-mL of iced-cold PBS for subsequent fluorescence measurements (F-2500, HITACHI, Tokyo, Japan). The excitation, emission, excitation slit, emission slit, and photomultiplier voltage were set at 395 nm, 507 nm, 5 nm, 5 nm, and 700 V, respectively. 1 mm fluorescence cuvette was used.

PHB Production in E. coli BL21P/pTOL03FphaCAB

25 ml of fresh LB media in a 250 ml glass flask was inoculated with an overnight culture of E. coli BL21P/pTOL03FphaCAB to reach an initial 0.D. 600 of 0.05. After 6-h cultivation, sterilized glucose and IPTG were added to the final concentration of 10 g/L and 15 nM, respectively. The pH of bacterial culture was measured at 12, 24,36, and 48 h and adjusted to 7 with 2 N HCl or 2 N NaOH when needed. Note that the genotype of E. coli BL21P is E. coli BL21(DE3)ΔptsG.

### PHB Quantification by Gas Chromatography (GC)

The gas chromatography (GC) method was used for PHB quantification. Briefly, freeze-dried cell (ca. 20 mg), obtained from the bacterial culture solution, was transferred to a clean spiral test tube and 1 mL of chloroform, 0.85 mL of methanol, and 0.15 mL of sulfuric acid were added. The tube was incubated in a water bath for 140 min at 80° C. and cooled down to room temperature. Then 1 mL of DI H₂O was mixed well by vortexing. After standing and layering, the organic phase was removed and filtered through a 0.2-nm PVDF filter and then analyzed by GC. The temperatures of the injector and detector were 230° C. and 275° C. respectively. The temperature of the column was set at 100° C. and increased to 200° C. at a rate of 10° C./min and maintained at 200° C. for 2 min. The PHB standard (363502) was purchased from Sigma-Aldrich. The PHB content was calculated as follows: PHB concentration (g/L)/biomass concentration (g/L)(%), where the biomass concentration was calculated by dividing freeze-dried cell weight by the culture volume for preparing freeze-dried cell.

### Results

AtxA is a Positive Regulatory Activator of E. coli Transcription

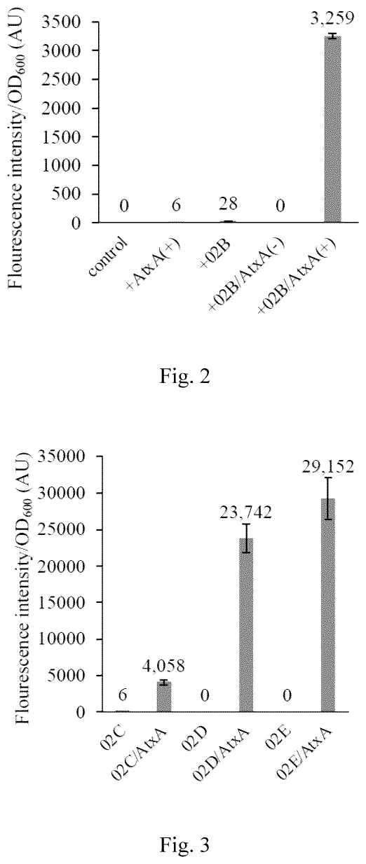

To examine the effect of AtxA on GFPᵤᵥ expression in E. coli, pTOL02B and pTOLAtxA were co-introduced into E. coli BL21(DE3). As shown in FIG. 2, BL21(DE3)/02B+AtxA had a significant specific fluorescence intensity (FI/OD₆₀₀) of 3,259±43 when IPTG was added to the final concentration of 0.03 mM. In contrast, the control experiments, including BL21(DE3) and BL21(DE3)/AtxA strains, showed no H. BL21(DE3)/02B and BL21(DE3)/02B+AtxA (no IPTG induction) showed FI/OD₆₀₀ of 28 and 0, respectively. FIG. 2 indicates that AtxA is a critical positive regulatory activator of transcription in E. coli.

### AT_{rich} Tract is a Negative Operator Site

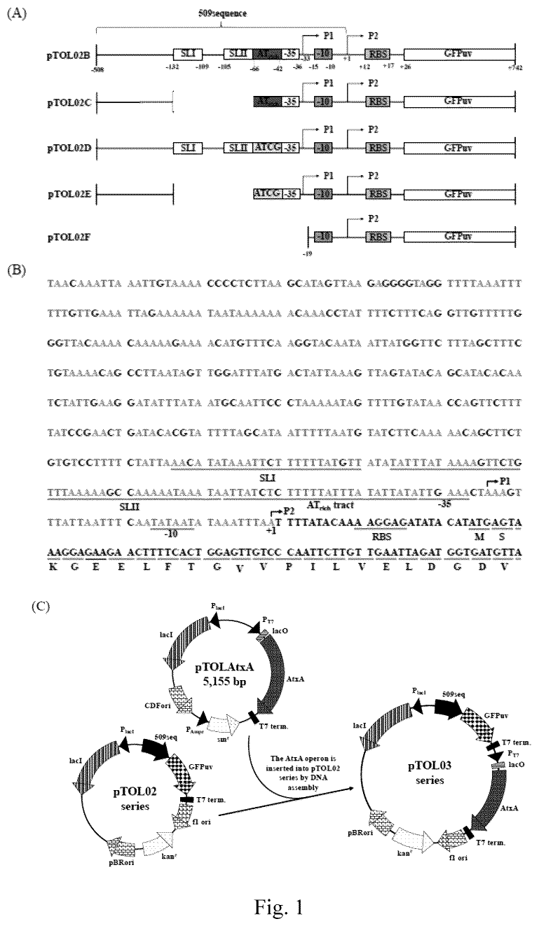

First, SLI and SLII were removed from pTOL02B to obtain pTOL02C. The Airier, tract in pTOL02B was replaced with a DNA fragment (ATCG_{avg}) that had the same length as a random sequence (56% AT, 14/25 bp, FIG. 1) to obtain pTOL02D. Then, pTOL02E was derived from pTOL02B by removing SLI and SLII and replacing the Airier, tract with ATCG_{avg}.

As shown in FIG. 3, BL21(DE3)/02C+AtxA had a FI/OD₆₀₀ of 4,058±294, which was comparable to that of BL21(DE3)/02B+AtxA. When Airier, in pTOL02B was replaced with the ATCG_{avg} fragment, BL21(DE3)/02D+AtxA exhibited a marked increase in the FI/OD₆₀₀ and reached 23,742±2,009. The effect of SLI/SLII removal and Airier, replacement on the AtxA regulated transcription was examined by monitoring the strain BL21(DE3)/02E+AtxA, which provided a FI/OD₆₀₀ of 29,152±2,864. It should be noted that 30 nM of IPTG was arbitrarily used for AtxA induction for the moment and the dependence of FI/OD₆₀₀ on the IPTG introduction will be investigated below to discuss the modularity of P_{AtxA} in responses to the IPTG.

Removal of the Upstream Site and −35 Sequence Revealed that AtxA Regulated Transcription E. coli is Independent of P1 and P2

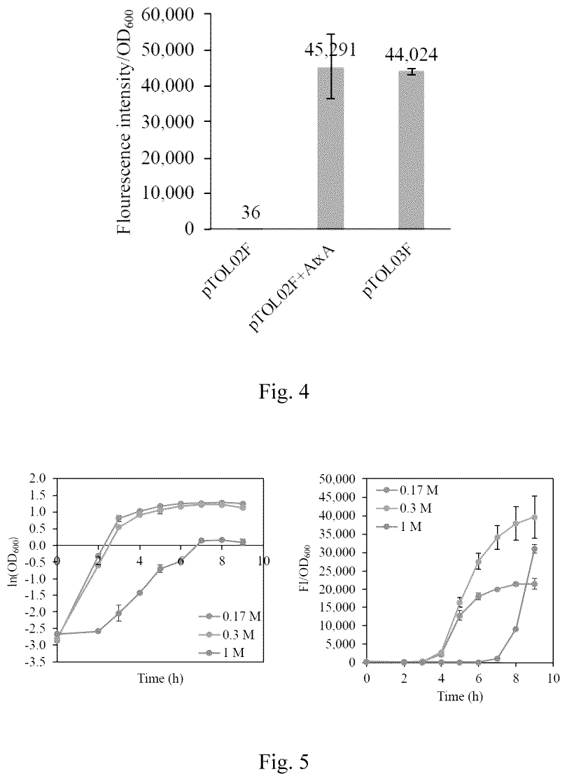

The effect of the upstream site and −35 sequence on AtxA regulated transcription was investigated by constructing pTOL02F. As shown in FIG. 4, BL21(DE3)/pTOL02F+AtxA had the highest FI/OD₆₀₀ of 45,291±8,879 among the pTOL02 series, which was 55% higher than that of pTOL02E+AtxA. Moreover, the removal of the upstream site and −35 sequence in pTOL02F showed that AtxA-regulated transcription is independent of P1 and P2. Besides, BL21(DE3)/pTOL02F had a low specific fluorescence of 36±6, indicating that the −10 sequence alone was a very weak promoter in E. coli.

The strong expression of GFPᵤᵥ in BL21(DE3)/pTOL02F+pTOLAtxA indicated that the AtxA regulated transcription involved three basic elements, which were AtxA, −10 sequence (TATA box), and E. coli cognate RNAP. The present invention further constructed pTOL03F by inserting the PT7-rbs-AtxA-ter operon into pTOL02F to verify the minimal elements for AtxA-regulated transcription in E. coli. FIG. 4 shows that BL21(DE3)/pTOL03F had a FI/OD₆₀₀ of 44,024±1,109 which was comparable to that of pTOL02F+pTOLAtxA. pTOL03D and pTOL03E were obtained by inserting the PT7-rbs-AtxA-ter operon into pTOL02D and pTOL02E, respectively.

AtxA-Regulated Transcription in E. coli is Stationary-Phase Specific

The cis-acting element required for AtxA-dependent GFPᵤᵥ expression simply consists of a −10 sequence, but no −35 sequence in E. coli. It can be concluded that AtxA-regulated transcription is independent of housekeeping σ⁷⁰. In fact, the −10 sequence (TATACT) exactly matched that of as-dependent promoter, also known as a type III promoter, which requires only the −10 sequence (TATACT) for transcription. The sigma factor σ^{s} is the starvation/stationary-phase sigma factor, and it is also known as a global regulator for responding to stress conditions, such as hyperosmolarity. The present invention demonstrated that AtxA-regulated transcription was stationary-phase specific and was suggested to be as-dependent.

To explore the effects of hyperosmotic conditions on the growth behavior and FI/OD₆₀₀ of E. coli, the growth behavior and FI/OD₆₀₀ of E. coli BL21(DE3)/pTOL03D were investigated in presence of 0.17, 0.30, and 1.00 M NaCl. As shown in FIG. 5A, 0.17 M NaCl (the recipe of the LB media) was optimal for bacterial growth, whereas 0.30 M had a slightly retarded growth. The 0.17 M and 0.30 M NaCl treatments had late-log-phase/stationary-phase transitions around a cultivation time of 3-4 h. Consistently, E. coli BL21(DE3)/pTOL03D started exhibiting fluorescence at 4 h (FIG. 5B). In contrast, 1.00 M NaCl had significantly impeded bacterial growth. Due to this, E. coli BL21(DE3)/pTOL03D reached the late-log-phase/stationary-phase transition at approximately 7 h. In accordance with the late-log-phase/stationary-phase transition, E. coli BL21(DE3)/pTOL03D showed no fluorescence until 7 h (FIG. 5B). The FI/OD₆₀₀ in the hyperosmolarity conditions of 0.30 M and 1.00 M at 9 h were 39,654±5,731 and 31,033±1,187, respectively, higher than that of the 0.17 M NaCl treatment (21,515±1,537).

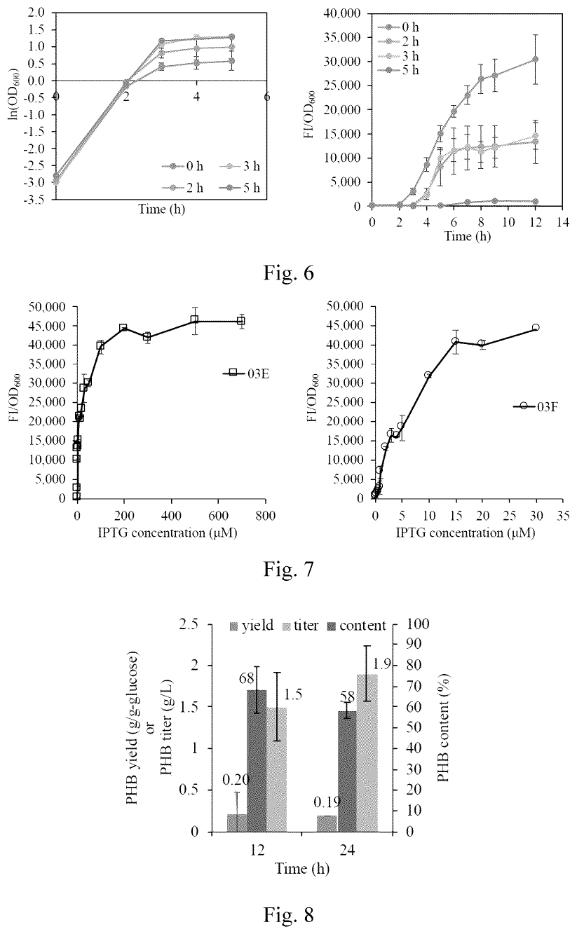

The stationary-phase specific protein expression was supported by investigating the AtxA induction time. When the supplementation of 0.03 mM IPTG for AtxA expression was supplemented at 2 and 3h, which is the time when E. coli BL21(DE3)/pTOL02D+pTOLAtxA reached the late-log-phase/stationary-phase transition stage (FIG. 6A), the fluorescence was revealed instantly (FIG. 6B). In contrast, IPTG supplementation at 0 h was a burden for bacterial growth and accelerated the onset of entry into the stationary phase (FIG. 6A). Consistently, the bacterial culture exhibited fluorescence at 3h, which was also in accordance with the stationary-phase expression. FIG. 6 shows that the induction at the stationary phase at h was too late for protein overexpression. While the AtxA and −10 sequence are two genetic elements for AtxA-regulated transcription, it is suggested that RNAP(as) is the cognate RNAP responsible for AtxA-regulated transcription.

### AtxA-Regulated Transcription is Tight-Control and Modulable

AtxA-regulated transcription was subsequently tested for its background expression by cultivating E. coli BL21(DE3)/pTOL03E and E. coli BL21(DE3)/pTOL03F without IPTG supplementation. Table 4 shows that both pTOL03E and pTOL03F had low FI/OD₆₀₀ of 155±1 and 333±10, respectively. Thereafter, the FI/OD₆₀₀ of both strains were investigated to determine the dependence on IPTG concentration. The FI/OD₆₀₀ of pTOL03E had a linear relationship with the IPTG concentration and reached a saturation value of 44,121±247 at 200 μM (FIG. 7A). Therefore, the induction/repression ratio was calculated as 285 (Table 3). The FI/OD₆₀₀ of pTOL03F also had a linear relationship with the IPTG concentration; however it was more sensitive to the IPTG conc., compared to that of pTOL03E (FIG. 7B). The FI/OD₆₀₀ showed a saturation value of at 15 μM of IPTG. pTOL03F had an induction/repression ratio of 132, in the same order as pTOL03E, whereas the IPTG dosage was lower by one order of magnitude (Table 3). The complete induction of pTOL03E and pTOL03F was accompanied by an acceptable OD₆₀₀ of 4.0±0.1 for both strains (Table 3). The induction/repression ratio of pTOL03F could be improved from 132 to 276 when cultured in M9 minimal medium supplemented with 20 g/L glucose (Table 3). A higher IPTG concentration (500 nM) was required for pTOL03F in the M9 minimal medium to reach full induction, and this can be attributed to the sterile medium for bacterial growth and protein overexpression. The performance of pTOL03E and pTOL03F as expression vectors was compared with that of E. coli BL21(DE3)/pDSK-GFPᵤᵥ. The plasmid pDSK-GFPᵤᵥ is conventional and known for its high green fluorescent protein (GFP) expression under the control of the promoter, P_{PsbA}, where P_{PsbA} is a constitutive promoter and can be 18 fold more efficient than PT7 The present invention, therefore, confirmed that the expression of GFPᵤᵥ in BL21(DE3)/pDSK-GFPᵤᵥ was growth-associated and evidenced that BL21(DE3)/pDSK-GFPᵤᵥ provided a peak FI/OD₆₀₀ of 22,878±3,742 at 8-8.5 h (Table 3). Although the induction/repression ratio cannot be defined for a constitutive promoter, the basal FI/OD₆₀₀ level of pDSK-GFPᵤᵥ could still be perceived when the bacterial culture first enters entered the log phase at 2-2.5 h, where the FI/OD₆₀₀ values were in a range of 1,747 and 6,805. The AtxA-regulated transcription had a better performance concerning strength and basal level expression compared to pDSK-GFPᵤᵥ.

TABLE 4
Performance of OD₆₀₀, FI/OD₆₀₀, and induction/repression ratio of E. coli BL21(DE3)/pTOL03E,
E. coli BL21(DE3)/pTOL03F, and E. coli BL21(DE3)/pDKS-GFPuv.

| | | | Specific | |
|---|---|---|---|---|
| | | | fluorescence | Induction/ |
| | IPTG induction | | intensity, | repression |
| plasmid/genetic trait | concentration, μM | OD₆₀₀*¹ | FI/OD₆₀₀*¹ | ratio |
| pTOL03E/ | 0 | 5.1 ± 0.0 | 155 ± 1 | 285 |
| upstream + ATCG_{avg} + (−35) + (−10) in | 200 | 4.0 ± 0.1 | 44,121 ± 247 | |
| rich medium | | | | |
| pTOL03F/ | 0 | 5.0 ± 0.0 | 333 ± 10 | 122 |
| (−10) in rich medium | 15 | 4.0 ± 0.1 | 40,597 ± 446 | |
| pTOL03F/ | 0 | 2.4 ± 0.0 | 98 ± 2 | 276 |
| (−10) in M9 medium | 500 | 2.1 ± 0.0 | 27,196 ± 176 | |
| pDSK-GFPuv/constitutive GFPuv | N.A. | 4.0 ± 0.1 | 22,878 ± 3,742 | N.A.*² |
| expression in rich medium | | | | |

*¹Samples were collected at 8-8.5 h for pDSK-GFPuv and at 9 h for pTOL03E and pTOL03F in the rich medium. Samples were collected at 12 h for pTOL03F in the minimal medium.
*²The induction/repression ratio cannot be defined for a constitutive promoter.

PHB production in E. coli BL21P/pTOL03Fpha CAB

To achieve the stationary-phase PHB production, E. coli BL21P/pTOL03phaFCAB was first aerobically cultivated in LB for 6 h, followed by the supplementation of glucose and IPTG to reach the concentrations of 10 g/L and 15 μM, respectively. It was shown in FIG. 8 that a PHB yield, content, and titer of g/g-glucose, 68±11%, and 1.5±0.4 g/L can be obtained at cultivation time of 12 h with the glucose consumption of 7.3±3.5 g/L (data not shown). When glucose was completely consumed, a PHB yield, content, and titer of 0.19±0.0 g/g-glucose, 58±4%, and 1.9±0.3 g/L can be obtained (shown in the time mark of 24 h in FIG. 8).

In the present invention, a 509-base pair DNA fragment [termed 509sequence, (−508)-(+1) relative to the P2 transcription start site] was cloned upstream of rbs-GFPᵤᵥ as pTOL02B to elucidate the AtxA regulated transcription. The 509sequence was dissected into the −10 sequence, −35 sequence, AT_{rich} tract, SLI/SLII, and the upstream site. In conjunction with the heterologous co-expression of AtxA (under the control of the T7 promoter), the −10 sequence (TATACT) was sufficient for AtxA-regulated transcription. Integration of pTOL02F+pTOLAtxA as pTOL03F showed that the AtxA-regulated transcription exhibited a strong specific fluorescence intensity (FI)/0D600 of 40,597±446 and an induction/repression ratio of 122. An improved induction/repression ratio of 276 was achieved by cultivating Escherichia coli/pTOL03F in M9 minimal medium. The newly developed promoter system termed P_{AtxA} consists of AtxA, the −10 sequence, and Escherichia RNA polymerase (RNAP). These three elements synergistically and cooperatively formed a previously undiscovered transcription system termed the promoter three-hybrid (P3H_{-10, AtxA}), which exhibited a tight-control, high-level, modulable, and stationary-phase specific transcription. The P_{AtxA} was used for phaCAB expression for the stationary-phase polyhydroxybutarate production and the results showed that a PHB yield, content, and titer of 0.20±0.27 g/g-glucose, 68±11%, and 1.5±0.4 g/L can be obtained. The positive inducible P_{AtxA}, in contrast to negative inducible, should be a useful tool to diversify the gene information flow in synthetic biology.

### DISCUSSION

It's widely known that the transcription of pagA (encoding PA toxin protein) in B. anthracis has two promoters, P1 and P2. It has been considered that the P1 promoter is subjected to AtxA regulation while the P2 promoter is constitutive. Some related arts have been conducted to elucidate the AtxA regulation mechanism, including biochemical studies of AtxA and identification of sequence- and/or structure-specific cis acting sites. Nevertheless, the complexity of pagA transcription makes results among literature inconsistent.

To avoid the complex and hierarchical regulation of pagA transcription in B. anthracis, trans-acting AtxA and the cis-acting 509 sequence, containing P1 and P2, were reconstituted in E. coli to study AtxA-regulated transcription and seek practical applications. In the present invention, AtxA was found to be a transcriptional activator in E. coli that actively facilitated transcription (as shown in pTOL02F+pTOLAtxA and pTOL03F).

The minimal AtxA-regulated promoter contains SLII, and SLII and AtxA binding is specific; however, the present invention showed that SLI/SLII removal increased the strength of the AtxA-regulated transcription. Those data suggested that SLII/AtxA binding actively downregulated the transcription activity in E. coli. This also suggested that the interaction between AtxA and Escherichia RNAP determines the AtxA-regulated transcription.

In the present invention, the promoters in pTOL02B and pTOL03B did not contain the downstream AT_{rich} tract; therefore, the effect of the downstream AT_{rich} tract on promoter activity could not be concluded. However, the present invention was the first to prove that the first AT_{rich} tract, more specifically, the 25-bp sequence upstream of the −35 box, is the most critical repressor operator. Removal of the first AT_{rich} tract from the 509 sequence greatly increased the AtxA-regulated transcription activity in E. coli (FIGS. 3 and 5). In general, the AT_{rich} tract can be a strong binding target for the histone-like nucleoid-structuring (H-NS) protein in E. coli. The binding of the AT_{rich} tract and H-NS protein prevents RNAP from binding to the promoter and is a common transcriptional control found in core gene transcription. AT_{rich} tract and H-NS protein binding can also act as a xenogeneic silencer, preventing the expression of horizontally acquired genes. In the present invention, the extreme burden of the AT_{rich} tract on the AtxA-regulated transcription in E. coli was governed by H-NS proteins through negative regulation.

In the present invention, the promoter, RNAP, and regulation factor were three typical elements that constitute the promoter system, the −10 sequence (TATACT), cognate RNAP, and AtxA exhibited a new transcription mechanism, where the synergistic and cooperative talks of three provided a tight-control, high-level, modulable, and stationary-phase specific expression promoter. The present invention hypothesizes a possible mechanism which involves two interactions. The first was that between Escherichia RNAP and −10 sequence, where the −10 sequence (TATACT) from Bacillus could barely be recognized by Escherichia RNAP. This became the basis for the tight-control of AtxA regulated transcription. The second is a possible interaction between Bacillus AtxA and Escherichia RNAP, in which AtxA acted as an activator for the resulting strong promoter activity. More specifically, the employment of the −10 sequence resulted in strong promoter activity specifically at the stationary phase. With the simplicity of the P_{AtxA}, which involves three transcriptional elements, the employment of P_{AtxA} in different hosts, following the central dogma of biology, is expected to have minimum toxicity and can be decoupled from the potential regulation restriction. Interestingly, most promoters used in synthetic biology are negative inducible, the positive inducible P_{AtxA} should be a useful tool to diversify the gene information flow in synthetic biology. While the orthogonality of hybridized holo-RNAP has been demonstrated that determines the initiation of the promoter activity at the certain cellular state, attention to the future development of P_{AtxA} can be genetically and biochemically focused on the activation mechanism of AtxA, or other PCVRs.

Another interesting DNA fragment of 509 sequence was the upstream site/−35 sequence, as seen in pTOL03E. The role of the upstream site/−35 sequence in AtxA-regulated transcription is clear, which titrates the AtxA activation activity.

Compared to pTOL03F, pTOL03E was less sensitive to the AtxA induction (FIG. 7). The comparable saturation values of the FI/OD₆₀₀ in pTOL03E and pTOL03F indicated that the titration effect of the upstream site/−35 sequence in pTOL03E followed the competitive kinetics (FIG. 7). Another result supporting the titration effect of the upstream site/−35 sequence is the low background of protein expression. While pTOL03F had an FI/OD₆₀₀ of 333±10 at 0 mM IPTG, pTOL03E had an FI/OD₆₀₀ of 155±1. An operator sequence with a titration function has found its application in the modulation of promoter activity. Note that pTOL03D, harboring SLI/SLII, had an FI/OD₆₀₀ of 160±4 at 0 mM IPTG (data not shown). The cognate RNAP in P_{AtxA} can rule out the dependence of σ⁷⁰ because the −35 sequence is not necessary for pTOL03F. Instead, it is the σ^{s} that is involved in P_{AtxA} for the following arguments. First, P_{AtxA} is a stationary-specific promoter. Second, the −10 sequence of TATACT used in P_{AtxA} is known as the type III promoter and can be recognized by σ^{s}. Third, the dependence of recombinant protein induction on high concentration of NaCl indicates the significant role of σ^{s}. The strong activity of P_{AtxA} confirms that sigma factors other than σ⁷⁰ can be used in applications. This is interesting because the intra-cellular concentrations of σ^{s} is one order of magnitude lower than that of the σ⁷⁰ at the log phase and is half of that of σ⁷⁰. Moreover, the in vitro data has shown that the binding affinity of σ^{s} to the core RNAP is only half of that of σ⁷⁰. The role of σ^{s} in P_{AtxA} should be thoroughly investigated, especially σ^{s} is a global regulator in the stationary phase.

Stationary phase protein expression is of interest in engineering applications, especially for toxic protein overexpression. However, promoters that can be used for stationary phase protein production often suffer from the low activity. Promoter activity at the stationary phase can be improved by random mutagenesis of the promoter region to isolate the mutant and the promoter can be increased by 16 fold to reach approximately 3,500 fluorescence intensity (GFPᵤᵥ)/0D600. The development of P_{AtxA} is one way to achieve an accurate and strong protein expression in the stationary phase while inflicting the minimum adverse effect on bacterial growth. For example, a high FI/OD₆₀₀ of 40,688±446 for E. coli BL21(DE3)/pTOL03F (FIG. 7) had a OD₆₀₀ of 4.0, which was comparable to the control experiments of E. coli BL21(DE3) in LB which had OD₆₀₀ of 4.1 at 9 h (data note shown).

In summary, a promoter three-hybrid has been developed, involving the −10 sequence (TATACT), Bacillus AtxA, and Escherichia RNAP. The promoter three-hybrid provides a tight-control, high-level, modulable, and stationary-phase specific transcription activity and was decoupled from the complex regulation restriction in B. anthracis. P_{AtxA} requires 15 uM IPTG for full promoter activity. A high induction/repression ratio of 230 was achieved when M9 minimal medium with g/L glucose was used. The 25-bp AT_{rich} tract was a strong negative operator for P_{AtxA} and SLI/SLII was a minor negative operator. The upstream site/−35 sequence may act as a decoy and titrate AtxA activity as a transcription activator.

In addition to the recombinant protein expression, the capability of P_{AtxA} was investigated in the application of metabolic engineering, as shown in FIG. 8. Bacterial culture at the stationary phase is considered a non-growth culture, yet glucose consumption and metabolisms still function. Secondary metabolites are specifically biosynthesized at the stationary phase and improved bioprocess productivity may be obtained when a non-growth culture is used. The present invention demonstrated a stationary-phase PHB production by using P_{AtxA} and the late addition of glucose at fermentation time of 6 h. The feasible results (FIG. 8) were in accordance with previous literature that non-growth cultures can be employed in biotechnology. Improved performance of E. coli BL21P/pTOL03FphaCAB for PHB production can be expected when the carbon flow can be directed to the PHB biosynthesis specifically in the stationary phase or employing fed-batch fermentation. One major advantage of stationary-phase bio-chemical production is that the bacterial growth and the heterologous metabolic pathway are decoupled. While a late addition of glucose was employed in the present invention, a parallel study was conducted where glucose was added at the beginning. The results showed that a low PHB yield of 0.10±0.0 g/g-glucose was obtained (data not shown). The low PHB yield resulted in a PHB content of 32±2%. The low PHB yield was because a significant amount of glucose was converted to acetic acid during the log phase (data not shown). In contrast, the high PHB yield of 0.19±0.0 g/g-glucose in FIG. 8 indicates that glucose was majorly converted into PHB during the stationary phase. In fact, the PHB content reported in the present invention was better than the one using a continuous promoter for PHB production using the same phaCAB cluster. This indicates that when the bacterial growth and heterologous metabolic pathway is decoupled, the balance of the bacterial growth and bio-based chemical production may be of secondary concern when constructing the microbial chassis. This concept can be studied in the future with P_{AtxA} developed in the present invention.

It will be understood that the above description of embodiments is given by way of example only and that various modifications may be made by those with ordinary skill in the art. The above specification, examples, and data provide a complete description of the present invention and use of exemplary embodiments of the invention. Although various embodiments of the invention have been described above with a certain degree of particularity, or with reference to one or more individual embodiments, those with ordinary skill in the art could make numerous alterations or modifications to the disclosed embodiments without departing from the spirit or scope of this invention.

What is claimed is:

1. A recombinant plasmid for manufacturing bio-based materials, comprising:
a promoter system; and
a rbs′-synthetic gene cluster,
wherein the promoter system comprises a Pribnow box and a transcriptional activator.

2. The recombinant plasmid of claim 1, wherein a method of producing the promoter system comprises:
(a) inserting a 509seq (SEQ ID NO: 29) into pET29a-EGFP to form a plasmid pTOL02A;
(b) replacing EGFP gene of pTOL02A with the GFPᵤᵥ gene to form the pTOL02B;
(c) deleting a SLI/SLII from the plasmid pTOL02B to form a plasmid pTOL02C;
(d) subjecting the plasmid pTOL02C to site-directed mutagenesis deletion to form a plasmid pTOL02F; and
(e) inserting the transcriptional activator into the plasmid pTOL02F to form a plasmid pTOL03F.

3. The recombinant plasmid of claim 2, comprising replacing an rbs-GFPᵤᵥ of the plasmid pTOL03F with the rbs′-synthetic gene cluster.

4. The recombinant plasmid of claim 3, wherein the rbs′-synthetic gene cluster comprises a rbs′-phaCAB gene cluster.

5. The recombinant plasmid of claim 2, wherein the site-directed mutagenesis deletion of the step (d) deletes −35, AT_{rich} tract, and an upstream site of the plasmid pTOL02C.

6. The recombinant plasmid of claim 1, wherein the transcriptional activator is an AtxA operon.

7. The recombinant plasmid of claim 6, wherein the AtxA operon is a PT7-rbs-atxA-T7 terminator.

8. The recombinant plasmid of claim 1, wherein the bio-based materials comprises polyhydroxybutyrate.

9. The recombinant plasmid of claim 1, wherein an activity of the promoter system is regulated by the transcriptional activator, the Pribnow box, and a cognate RNAP.
